(19) United States
(12) Patent Application Publication
(10) Pub. No.: US 2015/0168370 A1
(43) Pub. Date: Jun. 18, 2015

(54) Quantitative Interpretation of Cell Index Profiles

(71) Applicant: Mayo Foundation for Medical Education and Research, Rochester, MN (US)

(72) Inventors: Steven M. Offer, Rochester, MN (US); Robert B. Diasio, Rochester, MN (US); Natalie J. Wegner, Rochester, MN (US)

(21) Appl. No.: 14/398,168

(22) PCT Filed: Mar. 8, 2013
(86) PCT No.: PCT/US2013/029925

§ 371 Date: Oct. 31, 2014

Related U.S. Application Data
(60) Provisional application No. 61/640,898, filed on May 1, 2012.

(51) Int. Cl. G01N 33/483

(52) U.S. Cl. CPC G01N 33/483

(57) ABSTRACT

In some implementations, a computer-implemented method includes obtaining, by a computer system, cellular data from a real time cell analyzer; selecting one or more samples from the cellular data; identifying, for each of the one or more samples or for the cellular data as a whole, waypoints that define an analysis window; determining, by the computer system using cellular data from the analysis window, results for each of the one or more samples based on one or more metrics; and providing, by the computer system, information that identifies at least a portion of the results for the one or more samples.

### CROSS-REFERENCE TO RELATED APPLICATIONS

This application claims the benefit of U.S. Provisional Application Ser. No. 61/640,898, filed May 1, 2012. The disclosure of the prior application is considered part of (and is incorporated by reference in) the disclosure of this application.

### STATEMENT AS TO FEDERALLY SPONSORED RESEARCH

This invention was made with government support under grant CA116964 awarded by the National Institutes of Health. The government has certain rights in the invention.

### TECHNICAL FIELD

This document generally describes improved quantitative interpretation of cell index profiles.

### BACKGROUND

A real time cell analyzer (RTCA) has been developed to provide electrical impedance-based data that reflect different cellular parameters, including cell viability, cell number, and cell morphology. Such impedance measurements have been reported in arbitrary units of cell index (CI).

### SUMMARY

This document describes computer-based techniques for improving quantitative interpretation of cell index profiles.

In some implementations, a computer-implemented method includes obtaining, by a computer system, data from a real time cell analyzer; selecting one or more samples from the data; identifying, for each of the one or more samples or for the data as a whole, waypoints that define an analysis window; determining, by the computer system using data from the analysis window, results for each of the one or more samples based on one or more metrics; and providing, by the computer system, information that identifies at least a portion of the results for the one or more samples.

Such computer-implemented methods can include one or more of the following features. The one or more metrics can include an area under curve metric; and a first result for a first sample from the one or more samples can be determined based on an area under a portion of a curve for the first sample that plots measurements for the first sample over time within the analysis window. The one or more metrics can include a slope metric; and a first result for a first sample from the one or more samples can be determined based on a slope for a portion of a curve for the first sample that plots measurements for the first sample over time within the analysis window. The one or more metrics can include an area under curve per unit of time metric; and a first result for a first sample from the one or more samples can be determined based on i) an area under a portion of a curve for the first sample that plots measurements for the first sample over time within the analysis window, and ii) divided the unit of time. The unit of time can be an hour. A first waypoint from the waypoints can be defined based on a time at which a treatment was applied to the one or more samples. A second waypoint from the waypoints can be defined based on a period of time after the treatment was applied to the one or more samples. A first waypoint from the waypoints can be defined based on a time interval after treatment was applied to the one or more samples. A second waypoint from the waypoints can be defined based a time at which the one or more samples reached a maximum cell index value. A second waypoint from the waypoints can be defined based on a time at which one or more curves that are generated from and that correspond to the one or more samples had a maximum slope. A second waypoint from the waypoints can be defined based on a time at which one or more curves that are generated from and that correspond to the one or more samples had a minimum slope. The method can also include smoothing the data from the real time cell analyzer.

In some implementations, a computer system includes one or more computing devices, an interface of the one or more computing devices that is programmed to obtain data from a real time cell analyzer, and a waypoint unit that is programmed i) to select one or more samples from the data and ii) to identify, for each of the one or more samples or for the data as a whole, waypoints that define an analysis window. The computer system can further include a quantitative analysis unit that is programmed to determine, using data from the analysis window, results for each of the one or more samples based on one or more metrics and a data presentation unit that is programmed to provide information that identifies at least a portion of the results for the one or more samples.

Such a computer system can include one or more of the following features. The one or more metrics can include an area under curve metric; and a first result for a first sample from the one or more samples can be determined by the quantitative analysis unit based on an area under a portion of a curve for the first sample that plots measurements for the first sample over time within the analysis window. The one or more metrics can include a slope metric; and a first result for a first sample from the one or more samples can be determined by the quantitative analysis unit based on a slope for a portion of a curve for the first sample that plots measurements for the first sample over time within the analysis window. The one or more metrics can include an area under curve per unit of time metric; and a first result for a first sample from the one or more samples can be determined by the quantitative analysis unit based on i) an area under a portion of a curve for the first sample that plots measurements for the first sample over time within the analysis window, and ii) divided the unit of time. The unit of time can be an hour. A first waypoint from the waypoints can be defined by the waypoint unit based on a time at which a treatment was applied to the one or more samples. A second waypoint from the waypoints can be defined by the waypoint unit based on a period of time after the treatment was applied to the one or more samples. A first waypoint from the waypoints can be defined by the waypoint unit based on a time interval after treatment was applied to the one or more samples. A second waypoint from the waypoints can be defined by the waypoint unit based a time at which the one or more samples reached a maximum cell index value. A second waypoint from the waypoints can be defined by the waypoint unit based on a time at which one or more curves that are generated from and that correspond to the one or more samples had a maximum slope. A second waypoint from the waypoints can be defined by the waypoint unit based on a time at which one or more curves that are generated from and that correspond to the one or more samples had a minimum slope. The computer system can also include a data smoothing unit that is programmed to smooth the data from the real time cell analyzer.

In some implementations, a computer program product embodied in a computer readable storage device storing instructions that, when executed, cause one or more computing devices to perform operations that include obtaining data from a real time cell analyzer, selecting one or more samples from the data, and identifying, for each of the one or more samples or for the data as a whole, waypoints that define an analysis window. The operations can further include determining, using data from the analysis window, results for each of the one or more samples based on one or more metrics, and providing information that identifies at least a portion of the results for the one or more samples.

Such a computer program product can include one or more of the following features. The one or more metrics can include an area under curve metric; and a first result for a first sample from the one or more samples can be determined based on an area under a portion of a curve for the first sample that plots measurements for the first sample over time within the analysis window. The one or more metrics can include a slope metric; and a first result for a first sample from the one or more samples can be determined based on a slope for a portion of a curve for the first sample that plots measurements for the first sample over time within the analysis window. The one or more metrics can include an area under curve per unit of time metric; and a first result for a first sample from the one or more samples can be determined based on i) an area under a portion of a curve for the first sample that plots measurements for the first sample over time within the analysis window, and ii) divided the unit of time. The unit of time can be an hour. A first waypoint from the waypoints can be defined based on a time at which a treatment was applied to the one or more samples. A second waypoint from the waypoints can be defined based on a period of time after the treatment was applied to the one or more samples. A first waypoint from the waypoints can be defined based on a time interval after treatment was applied to the one or more samples. A second waypoint from the waypoints can be defined based a time at which the one or more samples reached a maximum cell index value. A second waypoint from the waypoints can be defined based on a time at which one or more curves that are generated from and that correspond to the one or more samples had a maximum slope. A second waypoint from the waypoints can be defined based on a time at which one or more curves that are generated from and that correspond to the one or more samples had a minimum slope. The operations can also include smoothing the data from the real time cell analyzer.

The details of one or more implementations are set forth in the accompanying drawings and the description below. Various advantages can be provided by certain implementations. For example, researchers can be benefited by reducing the subjectivity of RTCA data analysis. For instance, data can be analyzed in more objective manner by assigning waypoints to determine analysis windows. In another example, existing technology can be improved to provide better quantitative indicators that are representative of entire ranges or key biological aspects of collected RTCA data. In another example, data smoothing can be implemented to remove noise in generated data and produce a more accurate representation of trends within data. In another example, data transformation techniques can be used to reveal non-obvious trends in data. In a further example, mathematical normalization techniques can be used to provide reproducible techniques for comparing data between experiments. In another example, users can be provided with a better understanding of analyzed data and an increased level of transparency with regard to how data is analyzed through the integration of the described techniques into an accessible user interface.

Other features, objects, and advantages of the invention will be apparent from the description and drawings, and from the claims.

### DESCRIPTION OF DRAWINGS

FIG. 1 is an example graph that depicts a waypoint being defined based on a set time after culture treatment.

FIG. 2 is an example graph that depicts separate waypoints being defined for experiments.

FIG. 3 is an example graph that depicts waypoints being defined based on the maximum cell index.

FIG. 4 includes example graphs that depict waypoints being defined based on a maximum slope of a cell index.

FIG. 4A includes example graphs that depict waypoints being defined based on a minimum slope of a cell index.

FIG. 5 includes example graphs that depict waypoint being defined based on the Y-intercept of first derivative transformed CI.

FIG. 6 includes example graphs that depict data smoothing techniques applied to cell index profiles.

FIG. 7 includes example graphs that depict data smoothing techniques are applied to eliminate effects of unavoidable experimental disruptions.

FIG. 8 includes example graphs that depict data analysis using two globally defined waypoints.

FIG. 9 includes example graphs that depict data analysis using waypoints defined individually for each replicate.

FIG. 10 includes example graphs that depict data analysis using waypoints defined individually for each sample within a replicate.

FIG. 11 includes example graphs that depict the results of experiments in which two treatments were applied to cells.

FIG. 12 includes example graphs that depict results of experiments using an analysis window that is globally defined.

FIG. 13 includes example graphs that depict results of experiments using an analysis window.

FIG. 14 includes example graphs that depict results of experiments where waypoints were individually assigned for each sample.

FIG. 15 includes graphs that depict treatments having differences when waypoints are individually assigned for each sample.

FIG. 16 includes example graphs that depict quantitatively differentiating CI profiles.

FIG. 17 depicts an example system configured to perform the techniques described with regard to FIGS. 1-16.

FIG. 18 is a flowchart that depicts an example technique for analyzing data from an RTCA device.

FIG. 19 is a block diagram of example computing devices.

Like reference symbols in the various drawings indicate like elements.

### DETAILED DESCRIPTION

This document describes computer-based techniques for improving quantitative interpretation of cell index profiles.

Computer-based tools for analyzing and reporting RTCA data have allowed users to perform basic measurement calculations on CI data, including single-point based analysis, slope of a line between two time points (and derivative estimates of doubling time), and the maximum or minimum CI in a user-defined window of time. Doubling time can be the time it takes for a cell index to double in value. However, while some of these tools have allowed users to compare a series of datapoints in an analysis window, they have not provided an objective way for users to select waypoints (e.g., single time point at which cell index data has been collected) that define the analysis window. Within existing tools, waypoints have been defined globally for an entire experiment, which may restrict the quantitative potential of the analyses performed. Such computer-based tools have also lacked a way to normalize data for quantitative comparison of results with results of other experiments. This has restricted the quantitative nature of data produced and may result in any analysis that is performed using such data being largely subjective.

This document describes improved computer-based systems and techniques for defining measurable endpoints of kinetic data generated by an RTCA unit. Kinetic data can be information that is collected repeatedly over time. Endpoints can be numeric representations of analyzed data, such as numeric measurement values that are calculated according to predefined and validated analytic formulas and procedures based on CI data recorded repeatedly over time. Such endpoints can provide meaningful information regarding different aspects of cell function. In one example, an analysis window (time frame) can be defined based upon the mathematical properties of the CI profile and calculations can be performed in this analysis window to generate data expressed as a rate (e.g., area under curve per hour (AUC/hr)). An analysis window can be a time between waypoints. A CI profile can be a representation (e.g., graphical, mathematical expression) of cell index data plotted relative to time of collection. Such resulting data can be normalized and expressed in terms of a Z-score (e.g., data normalization strategy that may be defined as ([sample value]−[population average])/[standard deviation of population]) for comparison with data from replicate experiments. Taken together, these improved techniques can be used to perform mathematical normalization of quantitative findings that can allow results to be compared between individual experiments. Normalization can cause statistical errors in repeated measures of data to be isolated, which can allow the underlying characteristics of the data to be compared. Although these techniques are described with regard to RTCA data, they can be applied to other types of data to provide an empirical framework for obtaining quantitative experimental endpoint data from any kinetic assay.

A variety of endpoints can be calculated with regard to RTCA data, such as calculated doubling time, slope, time dependent IC50, CI at a time point vs. concentration, max CI in a time period vs. concentration, min CI in a time period vs. concentration, change in CI (max-min) in a time period vs. concentration, and/or area under curve in a time period vs. concentration. Area under curve can be the calculated area contained by a curve and the X axis of a plot in a given measurement window.

RTCA analysis software has offered a limited number of truly quantitative endpoints that fully utilize the power of the time course data obtained. For example, one of the disadvantages of currently available offerings for RTCA analysis is the lack of methodology to empirically establish waypoints that define the analysis window. Instead, waypoints have been defined in RTCA acquisition and analysis software using arbitrarily defined minimum and maximum times. Different treatments (e.g., experimental manipulation of cells that may include application of a chemical compound) within a given experiment may result in profoundly varied CI profiles. In such a case, arbitrarily defined waypoints may inappropriately skew data and may be of limited quantitative use. Additionally, a user may be unable to establish a normalization schema that allows data to be effectively compared to that obtained in other experiments. Overall, for many experimental situations, RTCA analysis software provides subjective information regarding data analysis, but not quantifiable data based on objective criteria.

RTCA analysis software has included limited analysis algorithms that have not allowed statistical waypoints to be assigned empirically and/or to be assigned using one or more standardized equation. Additionally, waypoint assignments have been performed on a global basis; such software has not allowed users to define different waypoints for different conditions within a data set. Furthermore, algorithms used in RTCA analysis software have not permitted users to normalize data using defined statistical waypoints, which has caused users to export data for analysis by other software programs. After exporting data, users may have to develop an algorithm to analyze their data in a meaningful way. In general, the use of arbitrarily defined waypoints has led to inter-assay variability making comparison of data from different experiments difficult.

This document describes techniques for resolving these and other deficiencies in RTCA analysis systems. For example, this document describes techniques for defining measureable quantitative endpoints for RTCA data, which can improve data analysis options and experiment comparison. RTCA analysis systems can be improved by establishing empirical techniques of defining waypoints on data collected from a single sample or from a group of samples, establishing additional quantitative and meaningful techniques for data analysis, the application of data normalization techniques to facilitate the analysis of replicate experimental data not collected in parallel, and/or developing a user-friendly integrated software package to perform data analysis using the techniques outlined herein.

To determine quantitative endpoints in kinetic data produced by an RTCA system, empirical analysis can be performed to establish waypoints along a profile, such as a CI profile. Waypoints can be a single time point at which data, such as cell index data, has been collected. A profile can include data measurements that are associated with times of collection, such as a CI profile that includes cell index data that is associated with times when the cell index data was collected.

With waypoints identified, quantitative analysis of the data bound by the waypoints can be performed to provide objective data for comparison across separate experiments. For example, RTCA cell index (CI) data can be provided in units defined as the area under the curve (AUC) between two waypoints per unit of time (referred to as area under curve per hour, AUC/hr). AUC between two waypoints, termed “a” and “b,” can be calculated using the following definite integral equation, which is an equation where f(x)dx represents CI data:

∫ₐ^{b}f(x)dx

### EQUATION 1

Since kinetic CI data is recorded as a series of individual data points instead of as single continuous equation, AUC can more simply be calculated using the trapezoid rule, an example of which is provided in the following equation:

$$\text{?} ( {x}_{k + 1} - {x}_{k} ) ( f ( {x}_{k + 1} + f ( {k}_{k} ) ) \\ \text{?} \text{indicates text missing or illegible when filed} \qquad EQUATION 2$$

In Equation 2, “a” equals a first waypoint and “N” equals the number of intervals between cell index values between “a” and a second waypoint. When AUC is calculated from a CI profile, the resulting value can be in units of “CI hours” (assuming hours were the unit of time used). When expressed in terms of hours, as AUC/hr, the units can be simply CI as (CI hours)/hours=CI. To more quantitatively portray CI data as a rate, data can be reported as CI/hr, which can be calculated as AUC/hr².

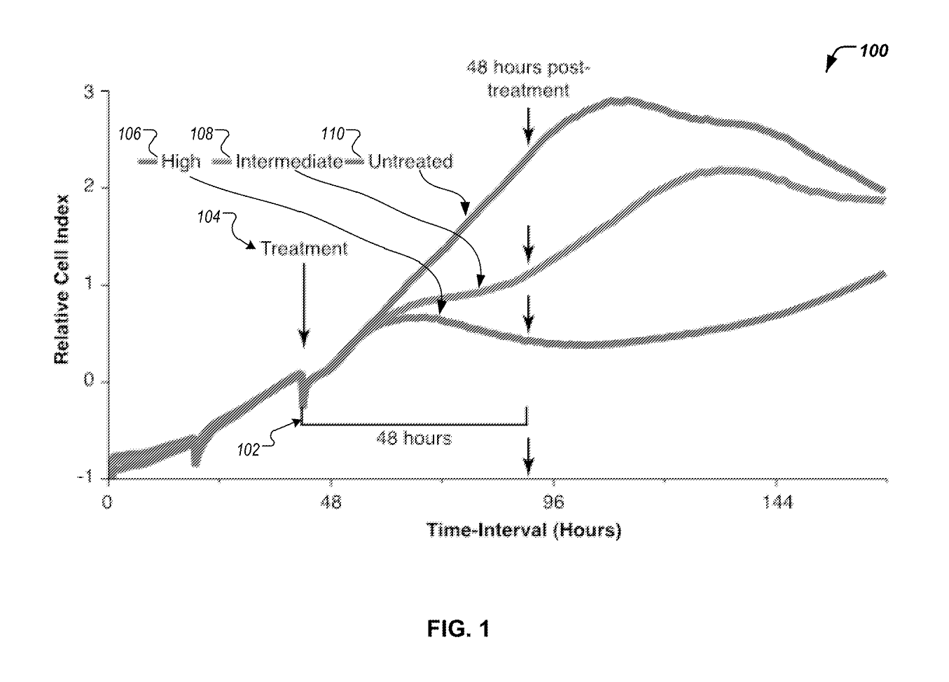

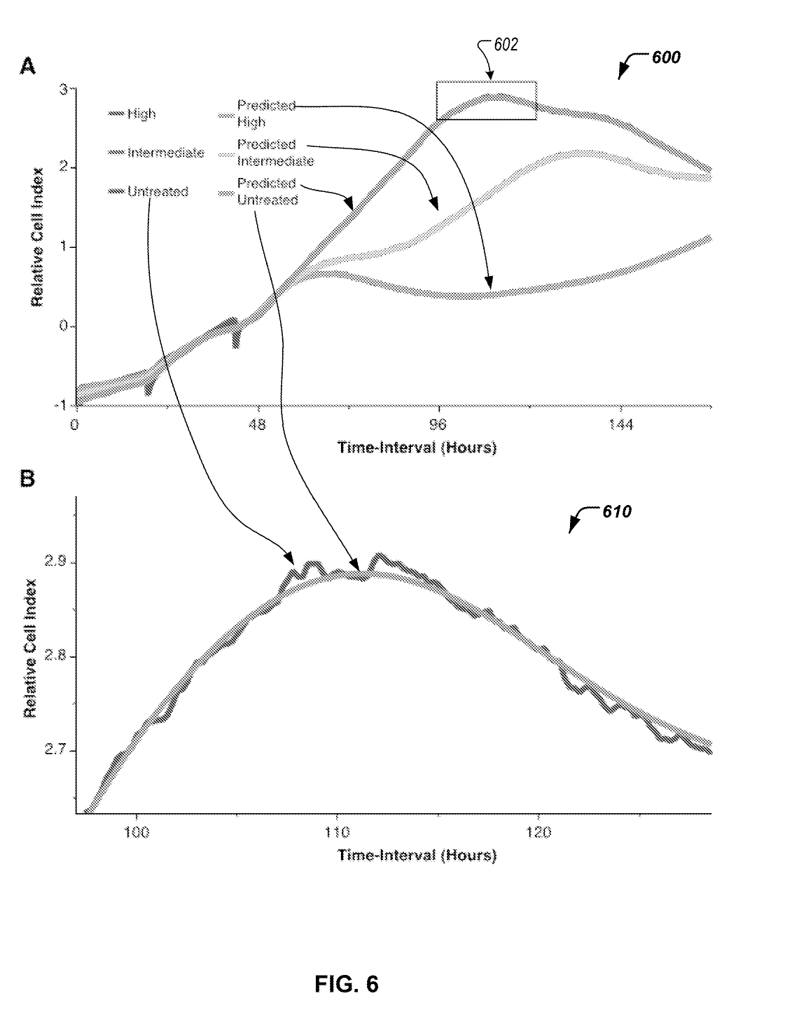

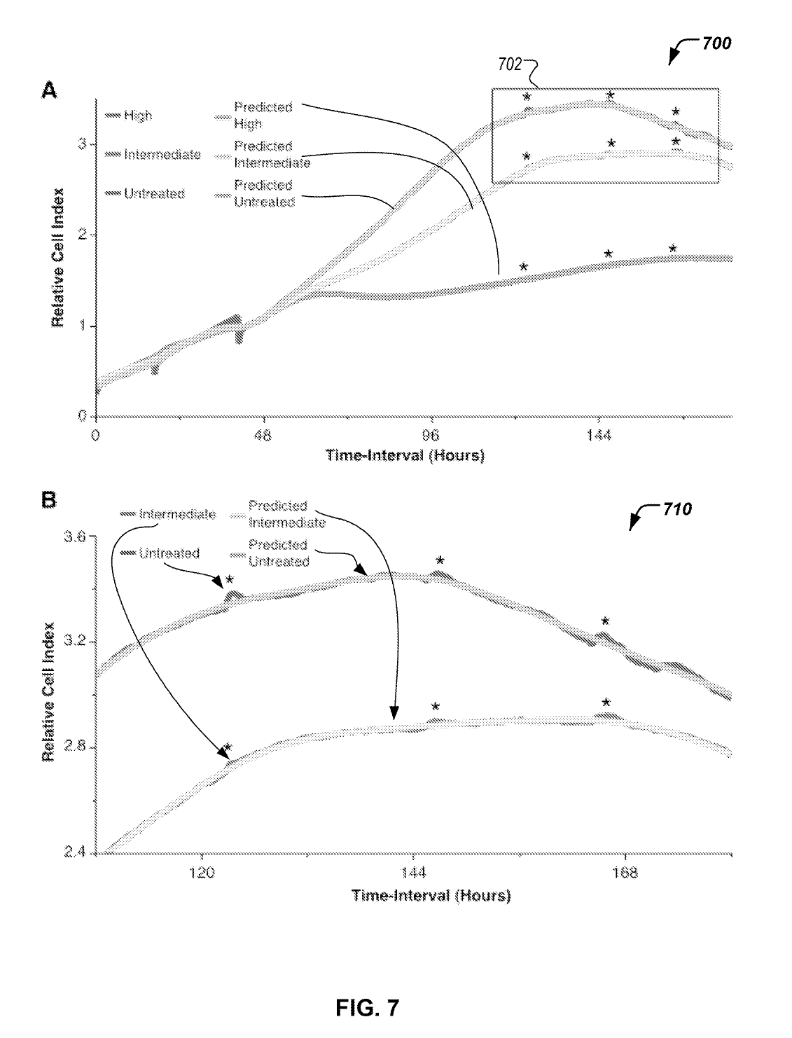

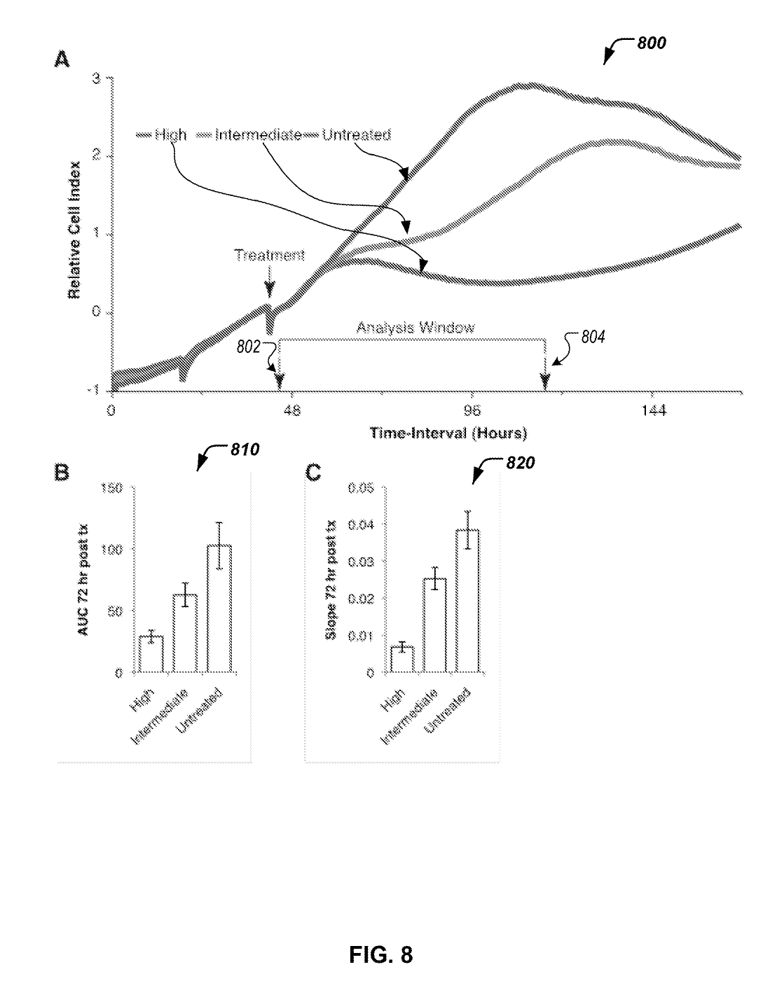

The description below regarding the figures is organized into three main parts. FIGS. 1-5 are described with regard to waypoint assignment, FIGS. 6-7 are described with regard to data smoothing, and FIGS. 8-16 are described with regard to quantitative analysis of kinetic data.

FIG. 1 is an example graph 100 that depicts a waypoint 102 being defined based on a set time after culture treatment 104. As depicted in FIG. 1, a waypoint 102 is defined for an experiment relative to a physical treatment 104 applied to the experiment. When the treatment 104 is applied to the entire experiment simultaneously, the waypoints can be the same across the entire experiment and can be considered a global waypoint. In the example experiment depicted in FIG. 1, HEK293T cells were plated in triplicate at time 0. After 40 hours of culture, cells were treated with two concentrations of a cytotoxic agent (a “high” treatment 106 and an “intermediate” treatment 108) or untreated 110 (media replaced with fresh media as a control). A cytotoxic agent can be an agent, such as a chemical compound, that is toxic to cells. Using this technique, waypoints can be globally defined and unilaterally apply to all CI curves in a given experiment.

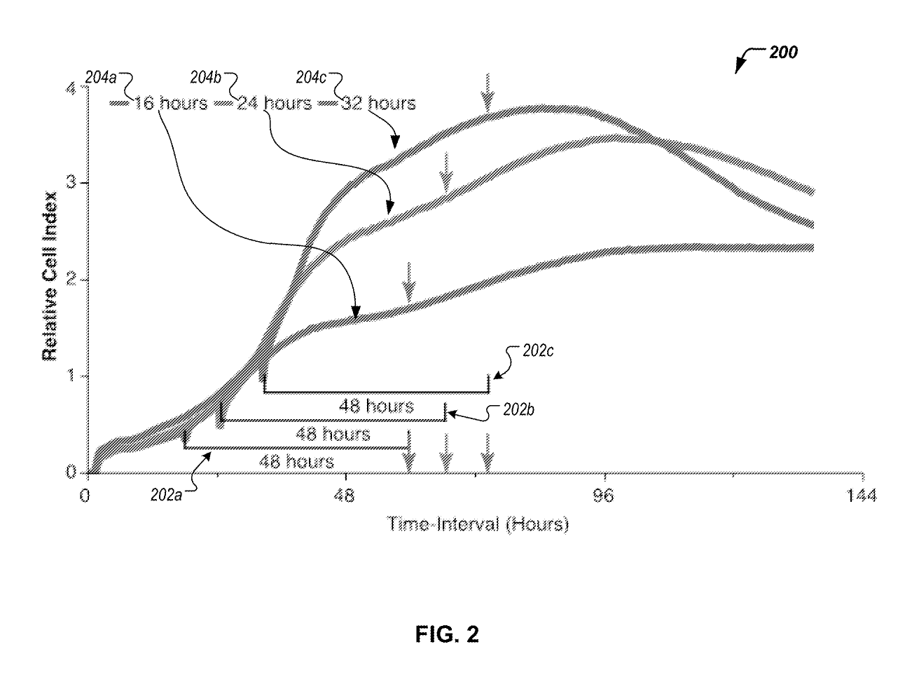

FIG. 2 is an example graph 200 that depicts separate waypoints 202a, 202b, and 202c being defined for experiments 204a, 204b, and 204c, respectively. The waypoints 202a-c are defined as a set time after treatment when samples for the experiments 204a-c were not treated concurrently. In the depicted example, treatment is applied at different times for three experiments. As depicted in the graph 200, treatment is applied at 16 hours for a first experiment 204a, at 24 hours for a second experiment 204b, and at 32 hours for a third experiment 204c. In the experiments 204a-c where treatments are applied at different times, the waypoint remains fixed at a set interval following treatment. If treatments occur at different times within an experiment, such as for experiments 204a-c, this technique allows waypoints to be defined for specific experiments where treatments were not simultaneous.

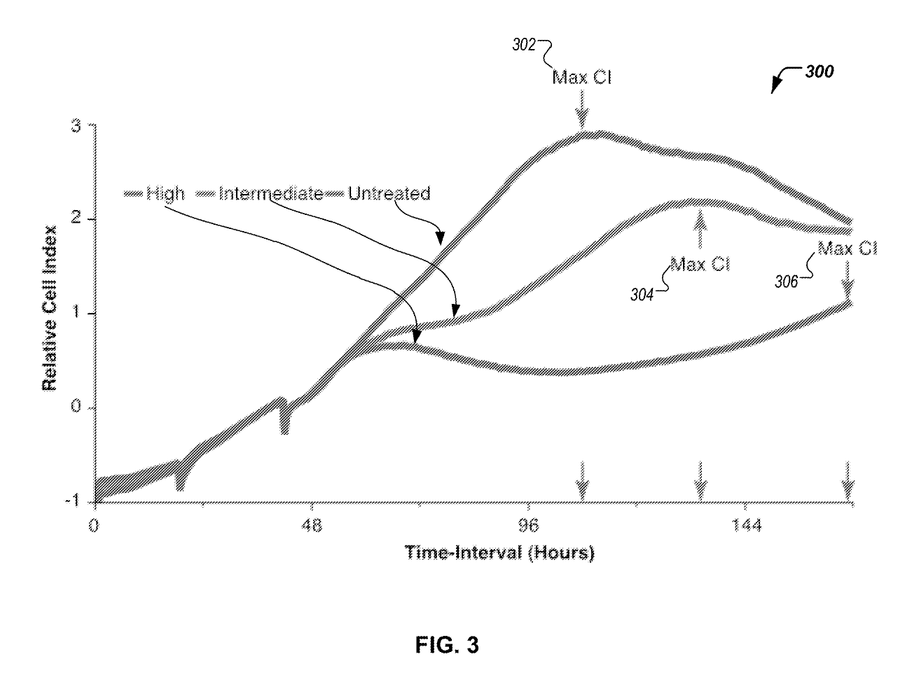

FIG. 3 is an example graph 300 that depicts waypoints being defined based on the maximum cell index. For example, waypoints can be assigned based upon the maximum CI readings 302-306. Waypoints can be assigned following treatment and/or independent of other treatments.

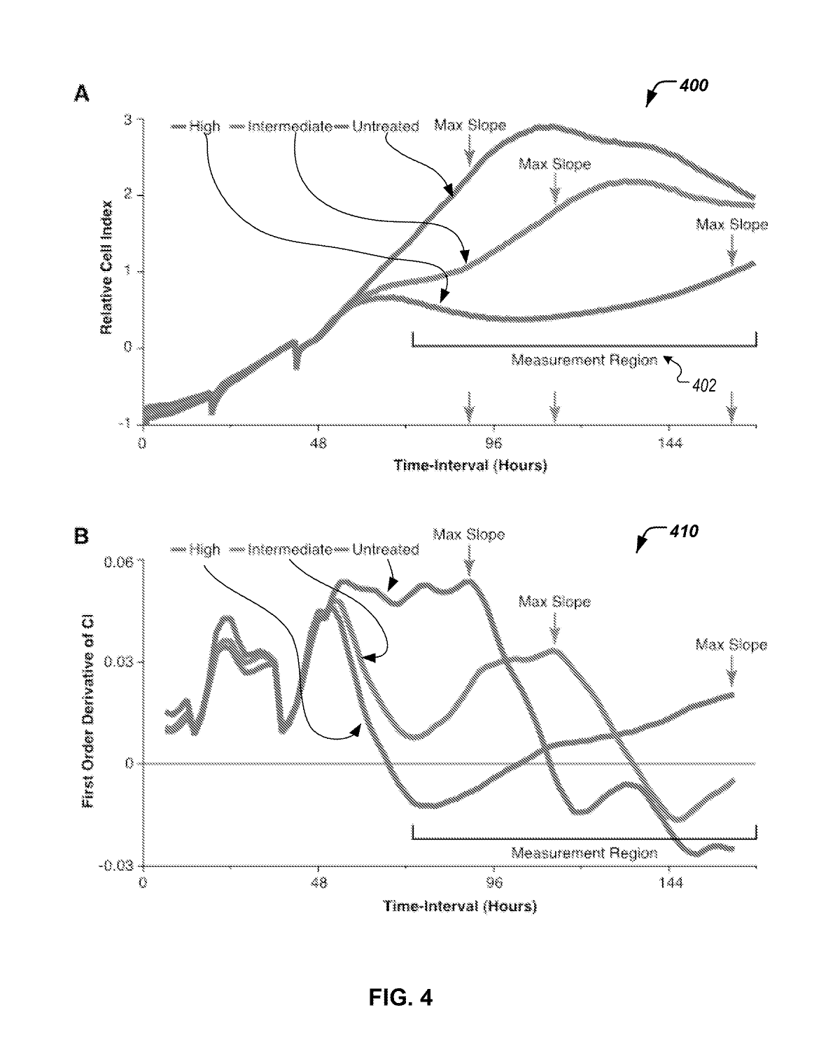

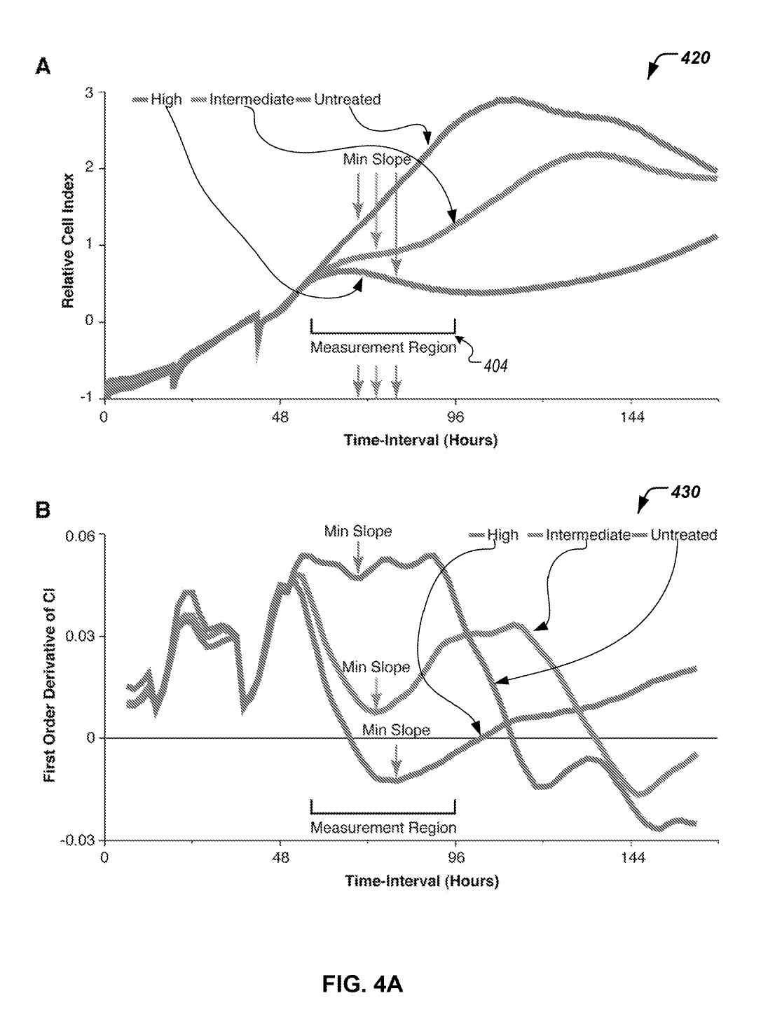

Depending upon the nature of the experiment, minimum CI could similarly be used to define waypoints. Waypoints could also be defined as the time at which the slope of the CI profile is greatest by determining slope as the first derivative of the CI. Such a technique for defining waypoints may indicate when cells are proliferating and/or expanding most rapidly. Waypoints could also be defined using first derivative data as the time at which the slope of the CI profile is the lowest. Such a technique for defining waypoints may indicate when cells are experiencing a toxic effect to treatment and when proliferation/expansion is at the lowest level. Additionally, this technique for defining waypoint may indicate when cells are experiencing the highest levels of cell death and/or detachment from the surface of the growth vessel. FIG. 4 includes example graphs A (400) and B (410) that depict waypoints being defined based on a maximum slope of a cell index. Graph A (400) depicts relative cell index curves. Graph B (410) depicts first order derivative plots that were calculated using linear regression of a 12-hour sliding window of data values and that represent the slope of the lines presented in graph A (400). A sliding window can include a technique whereby calculations are repeatedly performed over a specifically sized subset of data. The region of interest following separation of cell indices after treatment is indicated as the “Measurement region” 402 in graph A (400). The arrows identifying the “Max Slope” in graph A (400) indicate the time-interval of the maximum slope as determined in graph B (410). Assignments can be specific to a given treatment.

FIG. 4A includes example graphs A (420) and B (430) that depict waypoints being defined based on a minimum slope of a cell index. Graph A (420) depicts relative cell index curves. Graph B (430) depicts first order derivative plots that were calculated using linear regression of a 12-hour sliding window of data values and that represent the slope of the lines presented in graph A (420). The region of interest following separation of cell indices after treatment is indicated as the “measurement region” 404 in graph A (420). The arrows identifying the “Min Slope” in graph A (420) indicate the time interval of the minimum slope as determined in graph B (430). Assignments can be specific to a given treatment.

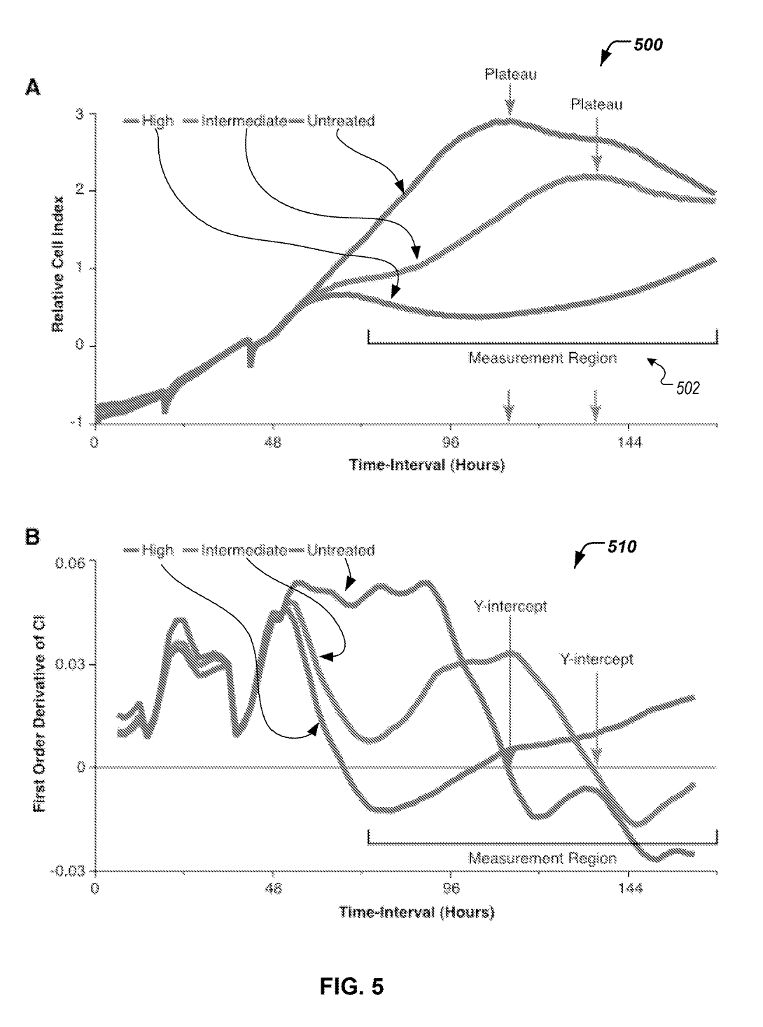

In another example, the first derivative data can also be used to define waypoint as the plateau of the CI profile. For instance, such a technique may provide result that would be similar to the maximum CI, however smoothing techniques can be applied when calculating slope that may result in selection of a slightly different waypoint. For example, calculating slope using a sliding window as described with regard to FIGS. 4 and 5 can impart some degree of smoothing, depending upon the size of the window used. FIG. 5 includes example graphs A (500) and B (510) that depict waypoint being defined based on the Y-intercept of first derivative transformed CI. Graph A (500) depicts relative cell index curves. Graph B (510) depicts first order derivative plots that were calculated using linear regression of a 12-hour sliding window of data values and that represent the slope of the lines presented in graph A (500). An example region of interest following separation of cell indices after treatment is identified as the “Measurement region” 502 in graph A (500). The plateau arrows depicted in graph A (500) identify the time-interval of the plateau (or maximum CI) as determined in graph B (510). Assignments can be specific to a given treatment.

FIG. 6 includes example graphs A (600) and B (610) that depict data smoothing techniques applied to cell index profiles. Data smoothing can include the generation of an approximation function to capture relevant patterns in the data and minimize effects of small scale data fluctuations (e.g., noise). RTCA is capable of collecting CI data in very small time intervals. A time interval can be spacing between collections of data or a collected data point and the first collected data point. In this experiment, data was collected every 15 minutes and cell index profiles were then smoothed using a spline function in JMP with A set equal to 1000. A spline function can be a piecewise polynomial function used for a variety of purposes, including data smoothing. Cell index profiles are presented in graph A (600) with superimposed smoothed profiles. The box 602 in graph A (600) represents a region of the curve that is expanded in graph B (610). Although some data can be lost in the smoothing process, the overall shape of the curve is unchanged. Spline smoothed data can be directly used for the determination of point slope as in FIG. 5 or FIG. 6 without the need for using a sliding window.

FIG. 7 includes example graphs A (700) and B (710) that depict data smoothing techniques are applied to eliminate effects of unavoidable experimental disruptions. To highlight the sensitivity of the RTCA, and to monitor effects of that subtle environmental changes may have on CI profiles, simulations were conducted to determine what happens when the incubator is opened to work with other cultures present therein. Cells were treated with various concentrations of a cytotoxic agent 40 hours after plating on the RTCA. At approximately 120, 144, and 168 hours, the door to the incubator containing cultures was opened and left ajar for 60 seconds before being reclosed. The change in environment due to this disruptions resulted in small “bumps” on the cell index profile, which are marked by asterisks (*) in graph A (700). These variations could result in erroneous estimates, such as an erroneous estimation of a maximum CI. The boxed region 702 in graph A (700) is expanded in graph B (710) to show the effect more clearly. A smoothing spline function was applied to the curves as in FIG. 6, and is denoted as the “predicted” lines in FIG. 7. As depicted in graph B (710), when compared to the predicted smoothed curve, the untreated sample appears to have actually plateaued prior to the door opening, which is the “true” maximum CI of the unsmoothed data. Spline smoothing of a dataset may reveal the true inflection point of a curve, which, in this example, is nearly 12 hours earlier. Data smoothing can be applied to the kinetic data obtained from RTCAs as a way to more accurately determine waypoints and/or analyze data using various attributes.

FIG. 8 includes example graphs A (800), B (810), and C (820) that depict data analysis using two globally defined waypoints 802 and 804. To test various quantitative analysis techniques, in this example cells were plated on an RTCA and treated with various concentrations of a cytotoxic agent after 40 hours of culture. CI data was collected at 15 minute intervals for approximately 168 hours in all. Referring to graph A (800), the waypoints 802 and 804 are defined at 2 hours post treatment and 72 hours post treatment. Experiments were performed in quadruplicate (a representative cell index profile is shown for a single replicate in graph A (800)) and analysis of these quadruplicate experiments are depicted in graphs B (810) and C (820). Cell index curves were normalized using a subtractive measure to the cell index at two hours after treatment. Mean values for measurement of area under the curve +/−standard deviation are presented in graph B (810). The slope of the line for the measurement area was determined for each sample using linear regression and is plotted +/−standard deviation of the 4 replicates, as depicted in graph C (820). Using this technique, both total area under curve, as depicted in graph B (810), and the slope between cell index values at the start and end of the analysis window, as depicted in graph C (820), showed differences for each of the three depicted treatments (high, intermediate, and untreated).

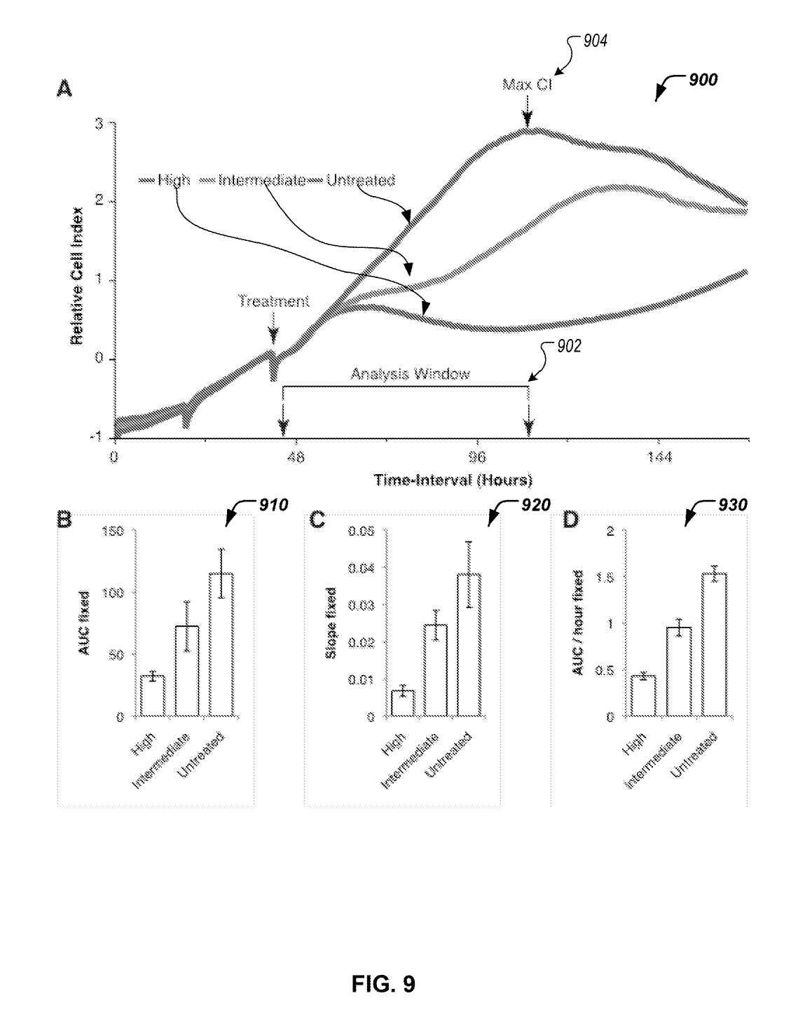

FIG. 9 includes example graphs A (900), B (910), C (920), and D (930) that depict data analysis using waypoints defined individually for each replicate. In the examples depicted in FIG. 9, experiments were performed in quadruplicate (a representative cell index profile is shown for a single replicate in graph A 900) and small differences in plating density between replicates were determined to cause the maximum cell index values to be reached at different times in each replicate. To control for such variation, the measurement region was changed to be specific to each replicate, as depicted in graph A (900). The start of the analysis window 902 was again defined as 2 hours after treatment, but the end was defined individually for each replicate as the time at which the untreated sample reached a maximum cell index value 904. This window 902 was applied globally across each replicate, but the size of the interval varied between replicates. Cell index curves were normalized using a subtractive measure to the cell index at two hours after treatment. Mean values for measurement of area under the curve +/−standard deviation are presented in graph B (910). The slope of the line for the measurement area was determined for each sample using linear regression and is plotted +/−standard deviation of the 4 replicates in graph C (920). Since the measurement region was variable between replicates, the AUC was normalized to the measurement region (time) and the resulting calculated values (AUC per hour) are presented +/−standard deviation for the four replicates in graph D (930). Using this technique, area under curve (AUC) and slope calculations revealed differences between the three treatments, as depicted in graph B (910) and graph C (920), respectively. Given the variable size of the analysis window, the AUC can be normalized for each replicate to the size of the time window, as depicted in graph D (930). The normalized AUC (AUC/hour) yielded the smallest error between replicates (compared to the relative size of standard deviation bounds in graphs B 910 and C 920).

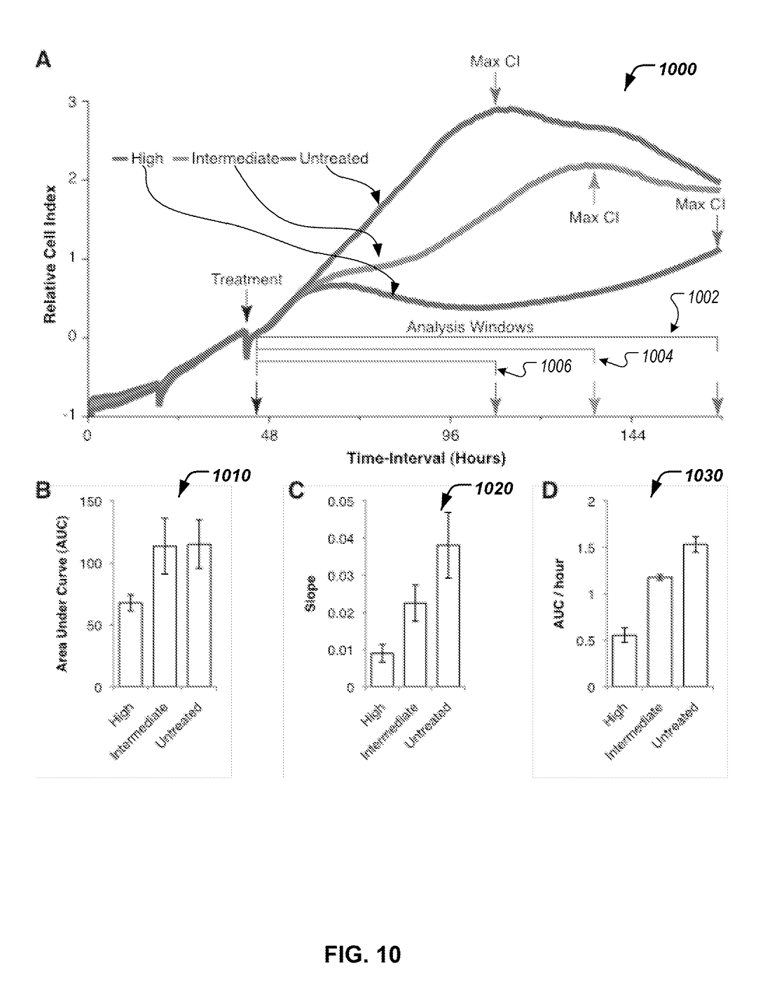

FIG. 10 includes example graphs A (1000), B (1010), C (1020), and D (1030) that depict data analysis using waypoints defined individually for each sample within a replicate. In the examples depicted in FIG. 10, samples were treated with various concentrations of a cytotoxic chemical that resulted in different shapes of the CI profiles and experiments were performed in quadruplicate (a representative cell index profile is shown for a single replicate in graph A 1000). A normalized AUC/hr calculation was applied to the entire data set by determining the second analysis waypoint individually for each sample (in contrast to determining a second waypoint for each replicate in FIG. 9). As depicted in graph A (1000), analysis windows 1002, 1004, and 1006 started two hours after treatment (a first waypoint defined globally) and ended at the time of maximum CI for each individual sample (a second waypoint defined individually for each sample within each replicate). The second waypoint was defined as the time at which the cell index was highest following treatment. As depicted in graph B (1010), total area under the curve in the measurement regions 1002-1006 was similar for intermediate treatment and untreated samples, suggesting that total area under the curve in this measurement region may be constant for samples which are capable of recovering from treatment and, as a result, area under the curve analysis may not be a useful technique for differentiating among treatments. The high treatment had a reduced total area in graph B (1010), however the maximum value was obtained at the last data point for the high treatment sample, as depicted in graph A (1000), and the profile had not yet reached plateau. As depicted in graph C (1020), linear regression across the measurement region (slope) is presented and accounts for the variable size of the analysis windows 1002-1006. As depicted in graph D (1030), normalization of the area under the curve by the time interval of the measurement region (AUC per hour) showed quantitative separation of the treatments and lower error than the other two analysis techniques depicted in graphs B (1010) and C (1020). The techniques using slope and AUC/hr, as depicted in graphs C (1020) and D (1030), accounted for the variable size of the analysis windows 1002-1006 and showed differences between the three treatments (high, intermediate, and untreated).

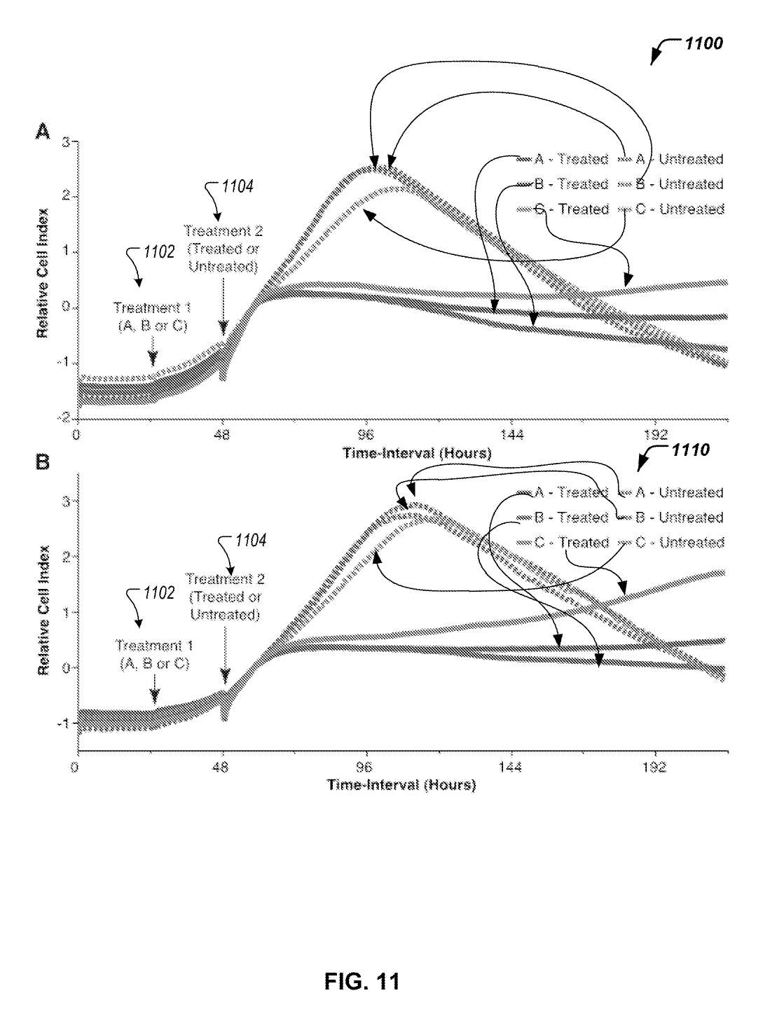

FIG. 11 includes example graphs A (1100) and B (1110) that depict the results of experiments in which two treatments were applied to cells. The graphs A (1100) and B (1110) depict profiles for two individual biological replicates of this experiment. 24 hours after plating, cells were treated with one of three compounds (A, B, or C), as indicated by Treatment 1 (1102). 24 hours later, half the cultures were treated with a cytotoxic chemical and the other half were given fresh media (untreated), as indicated by Treatment 2 (1104). As indicated in the graphs A (1100) and B (1110), cells that were treated with B or C and the cytotoxic agent did not show separation of CI profiles until after CI of untreated cultures had plateaued. The results depicted in graphs A (1100) and B (1110) indicate that changes in CI can happen on different time scales.

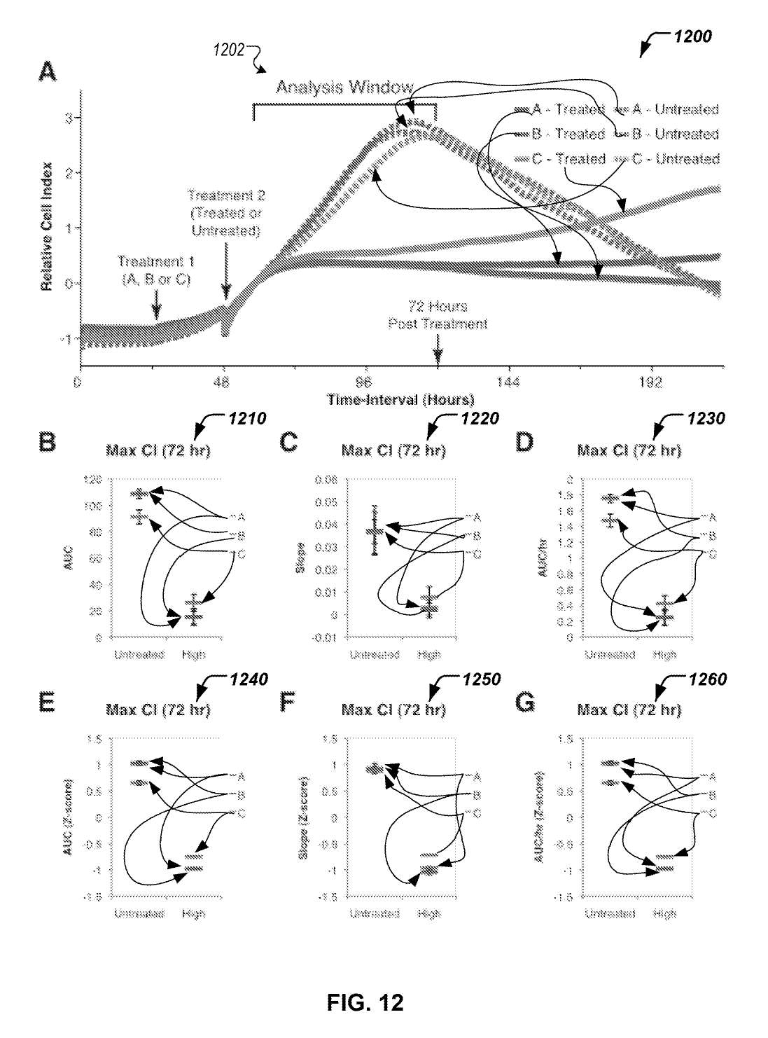

FIG. 12 includes example graphs A (1200), B (1210), C (1220), D (1230), E (1240), F (1250), and G (1260) that depict results of experiments using an analysis window 1202 that is globally defined as 10 hours post treatment to 72 hours post treatment. As depicted in graph A (1200), later responses to treatment, such as the differing responses to A—treated, B—treated, and C—treated after the 72 hours post treatment, may be missed if the analysis window 1202 is applied across all samples. Graph B (1210) depicts average +/−standard deviation of the two biological replicates for AUC, graph C (1220) depicts average +/−standard deviation of the two biological replicates for slope, and graph D (1230) depicts average +/−standard deviation of the two biological replicates for AUC/hr. As indicated by graphs B-D, using the analysis window 1202 did not reveal significant difference between A and B treatments that received the cytotoxic agent. To further reduce replicate variability Z-score normalization techniques were applied to datasets, the results of which are depicted in graphs E-G (1240-1260). Normalization by Z-score can be performed in a variety of ways, such as dividing the sample-population average by the population standard deviation. While standard deviation was reduced using Z-score normalization for all treatments, A and B receiving the cytotoxic drug did not show significant differences in graphs E-G (1240-1260).

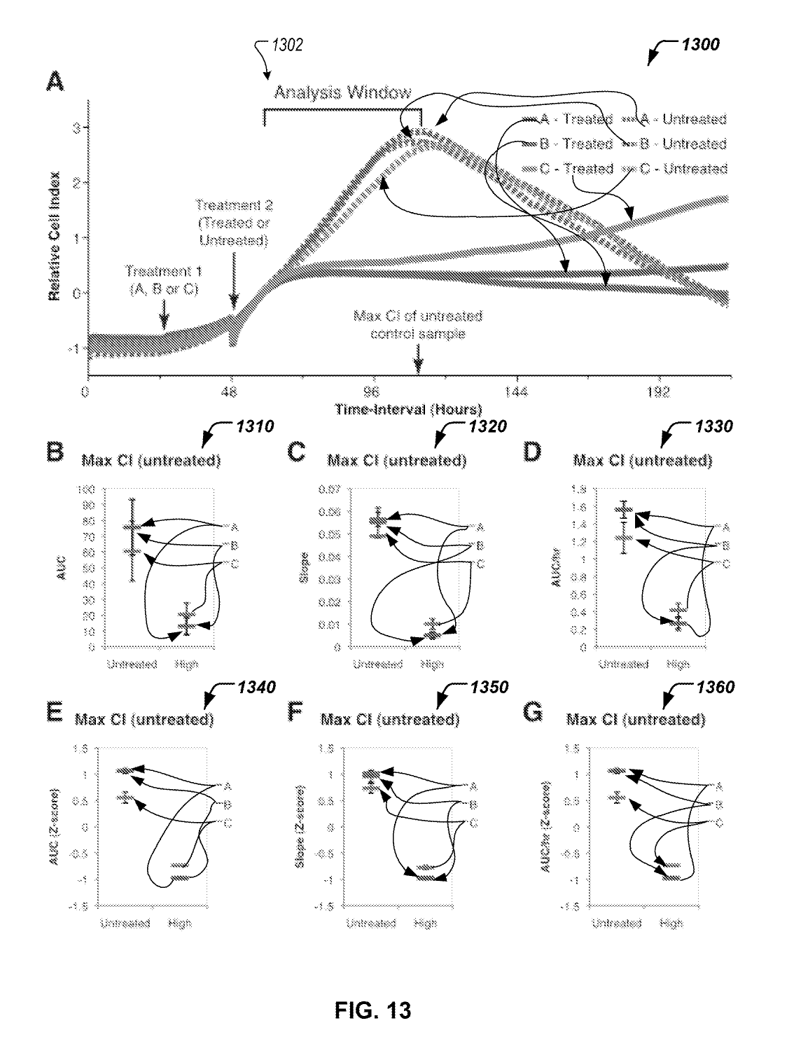

FIG. 13 includes example graphs A (1300), B (1310), C (1320), D (1330), E (1340), F (1350), and G (1360) that depict results of experiments using an analysis window 1302 defined as starting 10 hours after treatment and extending until the maximum CI for treatment A not receiving the cytotoxic chemical. As depicted in graph A (1300), later responses may not be captured by setting a fixed window based upon the cell index profile of a “control” treatment. Graphs B-D (1310-1360) depict the following: average +/−standard deviation of the two biological replicates for AUC (graph B 1310), slope (graph C 1320), and AUC/hr (graph D 1330). Results for each replicate were normalized by Z-score ( [sample-population average]/[population standard deviation]) and are presented in graphs E-G (1340-1360).

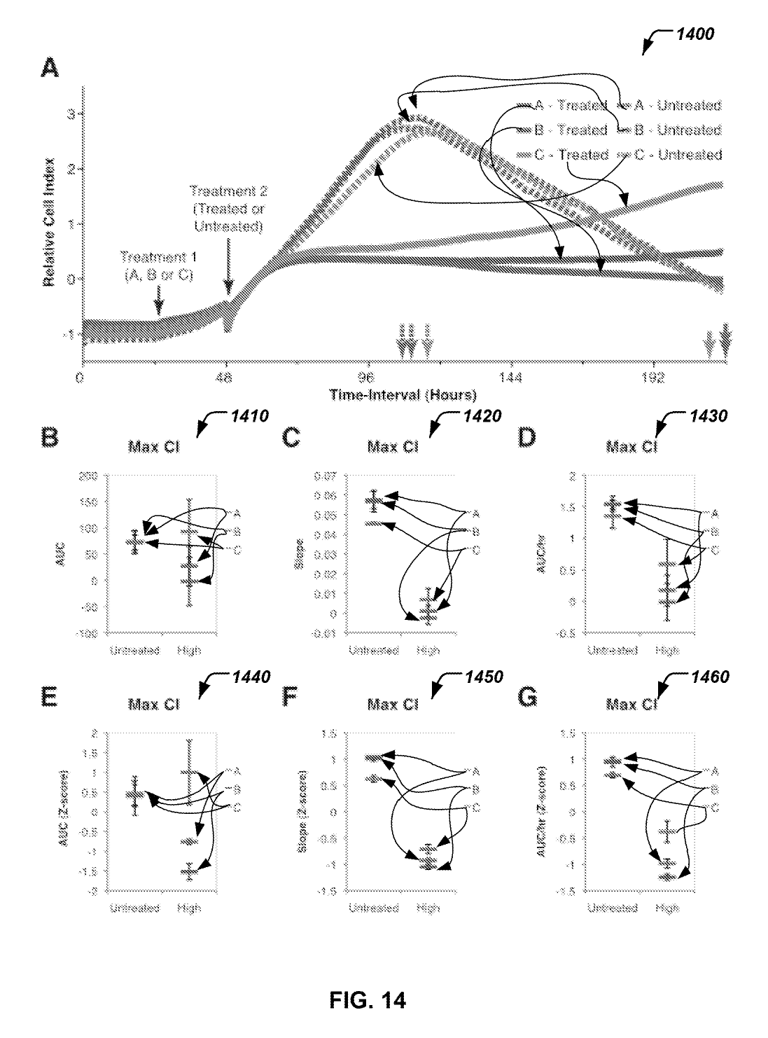

FIG. 14 includes example graphs A (1400), B (1410), C (1420), D (1430), E (1440), F (1450), and G (1460) that depict results of experiments where waypoints were individually assigned for each sample. Referring to graph A (1400), waypoints are individually assigned for each sample to define the analysis window as 10 hours post treatment to the maximum CI. By assigning waypoints dependent upon individual cell index profiles, a quantitative value can be assigned to all events in an experiment. The cell index curves of samples treated with the cytotoxic agent began separating from the untreated samples approximately 12-16 hours after treatment, suggesting a lag prior to the effects of the drug manifesting. Following this lag, some samples receiving the drug did not recover to the point that the slope of the cell index profile was positive, or to the point that the CI was higher than that at 16-24 hours post treatment. For these samples, the maximum CI was determined in a window established between 96 hours and the end of the experiment. In the event that the maximum CI was at 96 hours (e.g., the general slope of the line was negative), the minimum CI was used to establish the second waypoint. The analysis window can be defined individually for each sample as starting 10 hours after treatment and extending until time of maximum cell index (A). The minimum CI following treatment can be used as the second waypoint for samples receiving the cytotoxic drug that maintained a negative slope following a recovery phase (extending until approximately 96 hours). If slope became positive following recovery, but CI did not rise above those of the recovery phase, the maximum slope following the minimum (after recovery) can be used as the second waypoint.

Graphs B-D (1410-1430) depict the average +/−standard deviation of the two biological replicates for AUC (graph B 1410), slope (graph C 1420), and AUC/hr (graph D 1430). Results for each replicate were normalized by Z-score ([sample-population average]/[population standard deviation]) and are presented in graphs E-G (1440-1460). Using such a technique, the slope and AUC/hr both showed separation of the A and B treatments receiving the cytotoxic agent, as depicted in graphs C and D (1420 and 1430). Normalization using the Z-score method revealed that the greatest separation between A, B, and C receiving the cytotoxic agent was observed using the AUC/hr calculation, as depicted in graphs F and G (1450 and 1460). Treatments A and B (with cytotoxic drug) showed separation when waypoints were individually assigned to each sample in an experiment, further exemplifying the robustness of this technique.

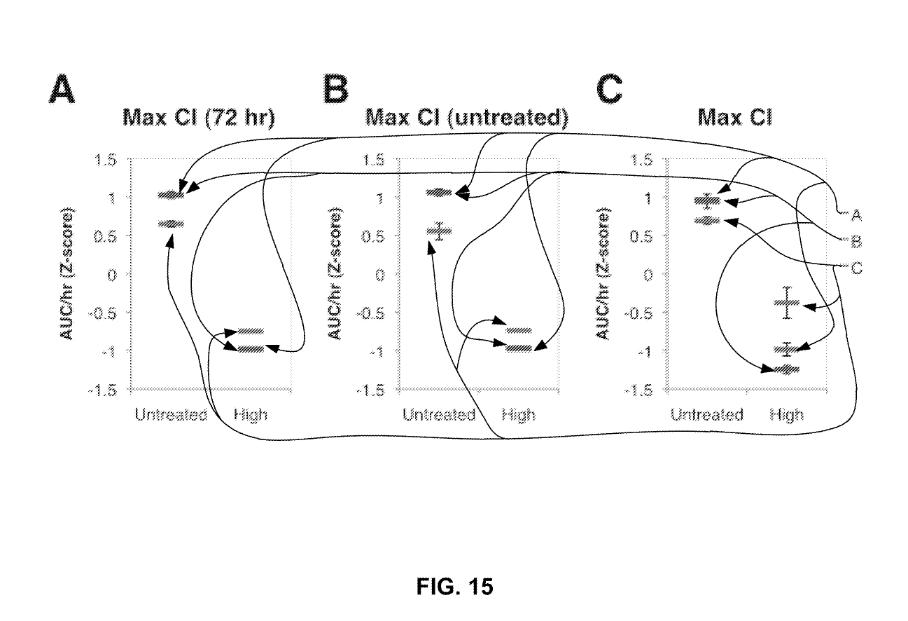

FIG. 15 includes graphs A-C that depict treatments A and B having differences when waypoints are individually assigned for each sample and data is analyzed using AUC/hr as a statistical endpoint.

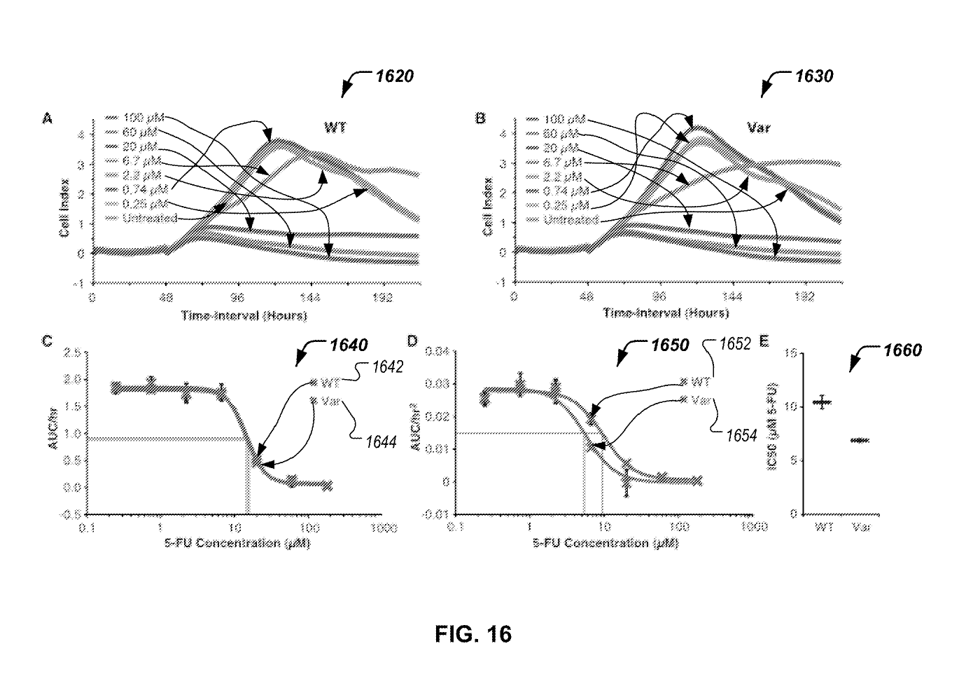

FIG. 16 includes example graphs A-E (1620-1660) that depict quantitatively differentiating CI profiles through the calculation of area under curve per hour squared (AUC/hr²). The graphs A-E (1620-1660) depict the results of an example experiment in which one of two treatments (compound A or compound B) were applied to cultures at approximately 24 hours after plating. At 48 hours, cells were treated with various concentrations of a cytotoxic agent (drug). Cell index profiles are presented in A and B (1620-1630) and a comparison of analysis methods for these data is presented in C and D (1640-1650).

To determine the sensitivity of the developed RTCA analysis techniques, example experiments were conducted using HEK293T/c17 cells that were plated at a density of 5,000 cells per well in E-plates at time 0 and treated with treatment A (depicted in graph A (1620)) or treatment B (depicted in graph B (1630)) 24 hours later. The example experiments included removing media containing treatment A or treatment B and replacing media with fresh media containing the indicated concentrations of the tested cytotoxic agent. CI was recorded for an additional 168 hours. CI values for each treatment were normalized to 4 hours post treatment with the cytotoxic agent, and AUC was determined in an analysis window defined independently for each treatment as 4 hours post treatment with cytotoxic agent to the time of maximum cell index. For samples exhibiting a negative slope after drug treatments began to affect CI values (approximately 72 hours), minimum CI was used to establish the second waypoint of the analysis window. The half maximal inhibitory concentration (IC₅₀) was determined for the cytoxic agent in cells treated with compound A (A, markers 1642) and compound B (B, markers 1644) using AUC/hr as a statistical endpoint, as depicted in graph C (1640). IC₅₀ determination was also performed determined for the cytotoxic agent in cells treated with compound A (A, markers 1652) and compound B (B, markers 1654) using AUC/hr² as a statistical endpoint, as depicted in graph D (1650). IC₅₀ can be a quantitative estimate of an amount of a particular substance that will inhibit a given biological process by half. Error bars in graphs C and D represent the standard deviation of three replicates performed in parallel. For comparative purposes, IC₅₀ was also determined using cellular viability 72 hours after treatment with the cytotoxic agent as a statistical endpoint, as depicted in graph E (1660). Viability was assessed using Cell Titer Blue reagent (manufacturer: Promega), and error bars in graph E (1660) represent the standard deviation of 4 replicate experiments performed in parallel.

As discussed above, HEK293T/c17 cells were treated with compound A or compound B, and response to a cytotoxic agent was monitored. The results of this monitoring are depicted in graph A (1620) and graph B (1630). As indicated in graph A (1620), treatment with less than 6.7 μM of the cytotoxic agent did not result in significant differences in CI profiles. For cells receiving compound A, 6.7 μM resulted in a reduced slope of the CI profile from 24 hours post treatment to plateau, and the plateau occurred at a later time than lower concentration treatments. As indicated in graph B (1630), cells treated with compound B saw a more profound change to the CI profile when treated with 6.7 μM of drug. A slight difference in CI profile was also noted between cultures receiving compound A or compound B treated with 20 μM of cytoxic agent. The CI profiles of cells receiving 60 or 100 μM of drug were similar regardless of which compound (A or B) they received.

To assess the potential application of the discussed techniques for CI data quantitation, AUC/hr was calculated for each treatment using an analysis window defined as 4 hours post-drug treatment to maximum cell index, as indicated in graph C (1640). In the case where slope of CI profiles following initiation of toxicity did not become positive (slope from approximately 72 hours onward), minimum CI was instead used to define the end of the analysis window. Whereas clear differences are noted for the cell index profiles of cells receiving compound A and compound B (mainly for 6.7 μM and 20 μM), these differences are not reflected in the calculated values. Additionally, AUC/hr for cells receiving 6.7 μM drugs was identified as not being significantly different than those values for lower concentration treatments. Using AUC/hr as a statistical endpoint, drug IC50 values for cells receiving compound A and compound B were found to be 15.0 μM and 13.8 μM, respectively.

The similarity of results of 6.7 μM treatments from lower concentration drug applications prompted an investigation of additional mechanisms for quantifying CI profile data. AUC/hr measures the mean area under the curve in the analysis window. While superficially this may appear to be a rate unit, AUC/hr can simplify to units of CI. To quantify the area in terms of rate units AUC/hr² was used, as depicted in graph D (1650). Using these techniques, IC₅₀ values for cells receiving compound A and compound B were determined to be 9.8 μM and 5.7 μM, respectively. As depicted in graph E (1660), those values are similar to those obtained for the drug using conventional methods of viability assessment, 10.4 and 6.9, respectively. AUC/hr² can more appropriately portray the overall differences in CI profiles between drug concentrations and pretreatment with compound A or compound B. Of analysis techniques, AUC/hr² can provide quantitative data that is most representative of the analysis window overall.

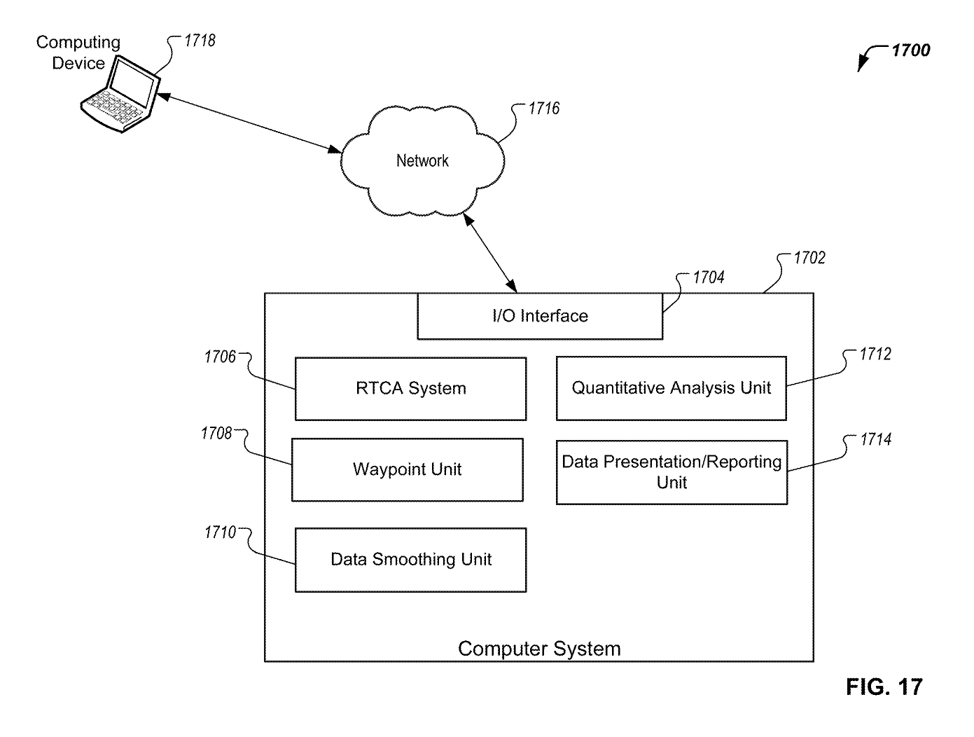

FIG. 17 depicts an example system 1700 configured to perform the techniques described with regard to FIGS. 1-15. The example system 1700 includes a computer system 1702, which can include one or more appropriate computing devices (e.g., a laptop computer, a desktop computer, a distributed computing system, a computer server, a cloud computing system). The computer system 1702 is depicted as including an input/output (I/O) interface 1704 through which the computer system 1702 communicates with other computing devices and/or users. For example, the I/O interface 1704 can be a wireless networking card through which the computer system 1702 wirelessly transmits to and/or receives data from other computing devices.

The computer system 1702 is depicted as including a real time cell analyzer (RTCA) system 1706, which can be a device that can provide electrical impedance-based data that reflect different cellular parameters, including cell viability, cell number, and cell morphology. In some implementations, the RTCA system 1706 is separate from and not included in the computer system 1702. In such implementations, the computer system 1702 can receive data from the RTCA system 1706, such as through the I/O interface 1704.

The computer system 1702 can also include a waypoint unit 1708 that is configured to identify one or more waypoints within data from the RTCA system 1706. The waypoints can be identified by the waypoint unit 1708 using one or more of the techniques described above with regard to FIGS. 1-5.

The computer system 1702 can also include a data smoothing unit 1710 that is configured to smooth data using one or more of the techniques described above with regard to FIGS. 6-7.

The computer system 1702 can also include a quantitative analysis unit 1712 that is configured to quantitatively analyze data from the RTCA system 1706 that has been smoothed by the data smoothing unit 1710 and using waypoints identified by the waypoint unit 1708. The quantitative analysis unit 1712 can perform such quantitative analysis using one or more of the techniques described above with regard to FIGS. 8-16.

The computer system 1702 can further include a data presentation/reporting unit 1714 to provide results from the quantitative analysis unit 1712 to any of a variety of users, such as a user of the computer system 1702 and/or a user of a separate computing device 1718 that is in communication with the computer system 1702 over a network 1716 (e.g., the Internet, a local area network, a wide area network, a wireless network, a virtual private network, and/or any combination thereof). The data presentation/reporting unit 1714 can provide data in any of a variety of appropriate formats, such as in the form of one or more of the graphs depicted in FIGS. 1-15. The RTCA system 1706, the waypoint unit 1708, the data smoothing unit 1710, the quantitative analysis unit 1712, and/or the data presentation/reporting unit 1714 can be implemented in any of a variety of appropriate forms, such as in hardware, software, firmware, or any combination thereof.

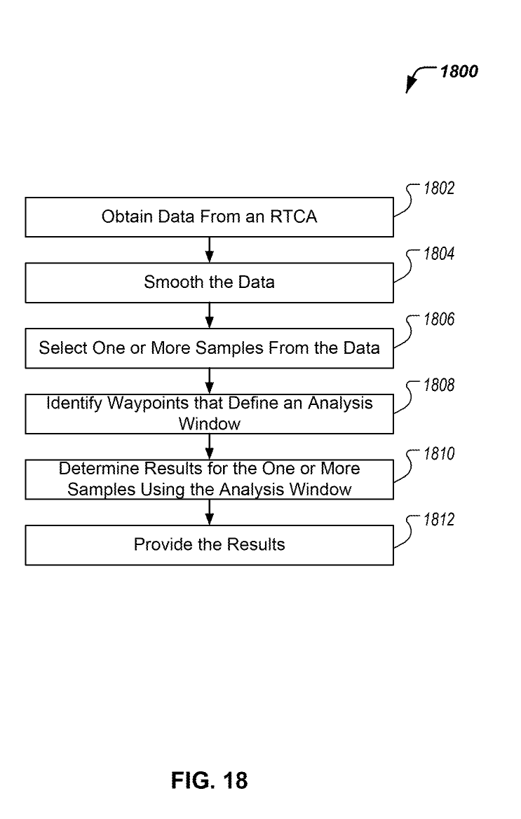

FIG. 18 is a flowchart that depicts an example technique 1800 for analyzing data from an RTCA device. The example technique 1800 can be performed by any of a variety of appropriate computing devices, such as the computer system 1702 described above with regard to FIG. 17.

At 1802, data is obtained from an RTCA device. For example, the computer system 1702 can obtain data from experiments performed on one or more samples from the RTCA unit 1706.

Data can be smoothed to eliminate anomalies from the data (1804). For example, the data smoothing unit 1710 of the computer system 1702 can use one or more of the techniques described above with regard to FIGS. 6-7 to smooth the data obtained from the RTCA unit 1706.

One or more samples can be selected from the data for analysis (1806). For example, samples 106, 108, and 110 as described above with regard to FIG. 1 can be selected.

Waypoints can be identified that define one or more analysis windows (1808). For example, a single global analysis window can be identified for the one or more samples, or individual analysis windows can be identified for each of the one or more samples. Various techniques can be used to identify waypoints, such as the techniques described above with regard to FIGS. 1-5.

Results can be determined for the one or more samples using the one or more identified analysis windows (1810). Various metrics can be used for determining and representing the results, such as AUC, slope, and/or AUC/hr. A variety of techniques can be used to determine results, such as those discussed above with regard to FIGS. 8-16.

The results can be provided (1812). For example, the computer system 1702 can provide the results through a user interface provided by the computer system 1702 (e.g., a graphical user interface provided on a display of the computer system 1702), can provide the results to a data storage device for use and/or retrieval at a later time, and/or can be provided to another computing device, such as the computing device 1718.

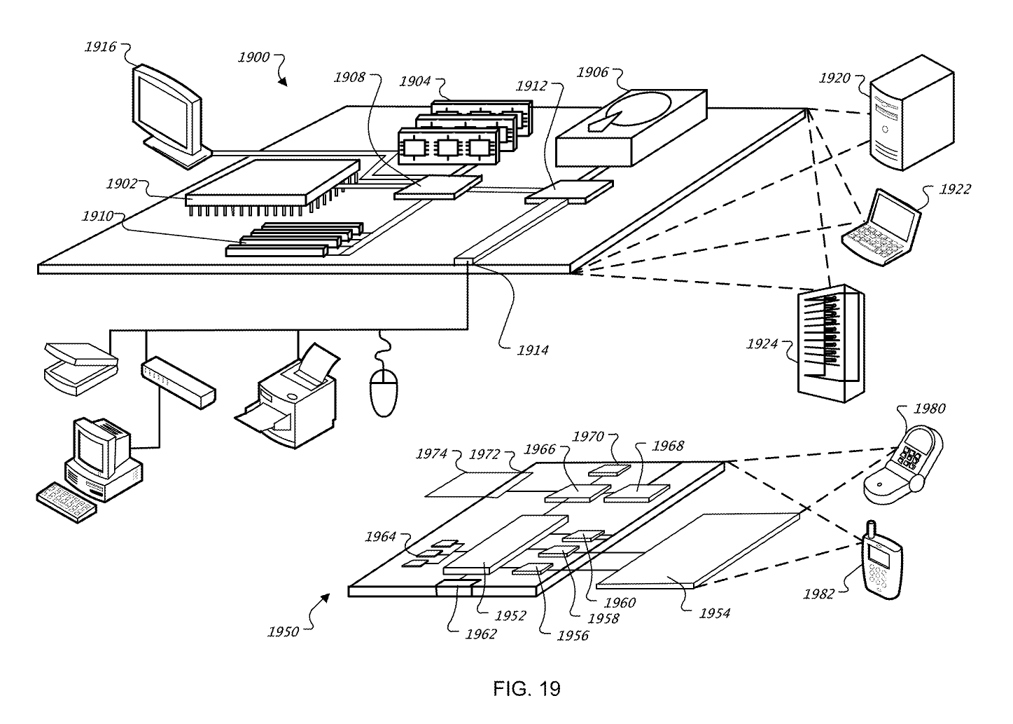

FIG. 19 is a block diagram of computing devices 1900, 1950 that may be used to implement the systems and methods described in this document, as either a client or as a server or plurality of servers. Computing device 1900 is intended to represent various forms of digital computers, such as laptops, desktops, workstations, personal digital assistants, servers, blade servers, mainframes, and other appropriate computers. Computing device 1950 is intended to represent various forms of mobile devices, such as personal digital assistants, cellular telephones, smartphones, and other similar computing devices. Additionally computing device 1900 or 1950 can include Universal Serial Bus (USB) flash drives. The USB flash drives may store operating systems and other applications. The USB flash drives can include input/output components, such as a wireless transmitter or USB connector that may be inserted into a USB port of another computing device. The components shown here, their connections and relationships, and their functions, are meant to be exemplary only, and are not meant to limit implementations described and/or claimed in this document.

Computing device 1900 includes a processor 1902, memory 1904, a storage device 1906, a high-speed interface 1908 connecting to memory 1904 and high-speed expansion ports 1910, and a low speed interface 1912 connecting to low speed bus 1914 and storage device 1906. Each of the components 1902, 1904, 1906, 1908, 1910, and 1912, are interconnected using various busses, and may be mounted on a common motherboard or in other manners as appropriate. The processor 1902 can process instructions for execution within the computing device 1900, including instructions stored in the memory 1904 or on the storage device 1906 to display graphical information for a GUI on an external input/output device, such as display 1916 coupled to high speed interface 1908. In other implementations, multiple processors and/or multiple buses may be used, as appropriate, along with multiple memories and types of memory. Also, multiple computing devices 1900 may be connected, with each device providing portions of the necessary operations (e.g., as a server bank, a group of blade servers, or a multi-processor system).

The memory 1904 stores information within the computing device 1900. In one implementation, the memory 1904 is a volatile memory unit or units. In another implementation, the memory 1904 is a non-volatile memory unit or units. The memory 1904 may also be another form of computer-readable medium, such as a magnetic or optical disk.

The storage device 1906 is capable of providing mass storage for the computing device 1900. In one implementation, the storage device 1906 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 1904, the storage device 1906, or memory on processor 1902.

The high speed controller 1908 manages bandwidth-intensive operations for the computing device 1900, while the low speed controller 1912 manages lower bandwidth-intensive operations. Such allocation of functions is exemplary only. In one implementation, the high-speed controller 1908 is coupled to memory 1904, display 1916 (e.g., through a graphics processor or accelerator), and to high-speed expansion ports 1910, which may accept various expansion cards (not shown). In the implementation, low-speed controller 1912 is coupled to storage device 1906 and low-speed expansion port 1914. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 1900 may be implemented in a number of different forms, as shown in the figure. For example, it may be implemented as a standard server 1920, or multiple times in a group of such servers. It may also be implemented as part of a rack server system 1924. In addition, it may be implemented in a personal computer such as a laptop computer 1922. Alternatively, components from computing device 1900 may be combined with other components in a mobile device (not shown), such as device 1950. Each of such devices may contain one or more of computing device 1900, 1950, and an entire system may be made up of multiple computing devices 1900, 1950 communicating with each other.

Computing device 1950 includes a processor 1952, memory 1964, an input/output device such as a display 1954, a communication interface 1966, and a transceiver 1968, among other components. The device 1950 may also be provided with a storage device, such as a microdrive or other device, to provide additional storage. Each of the components 1950, 1952, 1964, 1954, 1966, and 1968, are interconnected using various buses, and several of the components may be mounted on a common motherboard or in other manners as appropriate.

The processor 1952 can execute instructions within the computing device 1950, including instructions stored in the memory 1964. The processor may be implemented as a chipset of chips that include separate and multiple analog and digital processors. Additionally, the processor may be implemented using any of a number of architectures. For example, the processor 1952 may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 1950, such as control of user interfaces, applications run by device 1950, and wireless communication by device 1950.

Processor 1952 may communicate with a user through control interface 1958 and display interface 1956 coupled to a display 1954. The display 1954 may be, for example, a TFT (Thin-Film-Transistor Liquid Crystal Display) display or an OLED (Organic Light Emitting Diode) display, or other appropriate display technology. The display interface 1956 may comprise appropriate circuitry for driving the display 1954 to present graphical and other information to a user. The control interface 1958 may receive commands from a user and convert them for submission to the processor 1952. In addition, an external interface 1962 may be provide in communication with processor 1952, so as to enable near area communication of device 1950 with other devices. External interface 1962 may provide, for example, for wired communication in some implementations, or for wireless communication in other implementations, and multiple interfaces may also be used.

The memory 1964 stores information within the computing device 1950. The memory 1964 can be implemented as one or more of a computer-readable medium or media, a volatile memory unit or units, or a non-volatile memory unit or units. Expansion memory 1974 may also be provided and connected to device 1950 through expansion interface 1972, which may include, for example, a SIMM (Single In Line Memory Module) card interface. Such expansion memory 1974 may provide extra storage space for device 1950, or may also store applications or other information for device 1950. Specifically, expansion memory 1974 may include instructions to carry out or supplement the processes described above, and may include secure information also. Thus, for example, expansion memory 1974 may be provide as a security module for device 1950, and may be programmed with instructions that permit secure use of device 1950. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 1964, expansion memory 1974, or memory on processor 1952 that may be received, for example, over transceiver 1968 or external interface 1962.

Device 1950 may communicate wirelessly through communication interface 1966, which may include digital signal processing circuitry where necessary. Communication interface 1966 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1968. In addition, short-range communication may occur, such as using a Bluetooth, WiFi, or other such transceiver (not shown). In addition, GPS (Global Positioning System) receiver module 1970 may provide additional navigation- and location-related wireless data to device 1950, which may be used as appropriate by applications running on device 1950.

Device 1950 may also communicate audibly using audio codec 1960, which may receive spoken information from a user and convert it to usable digital information. Audio codec 1960 may likewise generate audible sound for a user, such as through a speaker, e.g., in a handset of device 1950. Such sound may include sound from voice telephone calls, may include recorded sound (e.g., voice messages, music files, etc.) and may also include sound generated by applications operating on device 1950.

The computing device 1950 may be implemented in a number of different forms, as shown in the figure. For example, it may be implemented as a cellular telephone 1980. It may also be implemented as part of a smartphone 1982, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

Although a few implementations have been described in detail above, other modifications are possible. Moreover, other mechanisms for improving quantitative interpretation of cell index profiles may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

1. A computer-implemented method comprising:
obtaining, by a computer system, cellular data from a real time cell analyzer;
selecting one or more samples from the cellular data;
identifying, for each of the one or more samples or for the cellular data as a whole, waypoints that define an analysis window;
determining, by the computer system using cellular data from the analysis window, results for each of the one or more samples based on one or more metrics, the results indicating at least one characteristic of the cellular data within the analysis window; and
providing, by the computer system, information that identifies at least a portion of the results for the one or more samples.

2. The computer-implemented method of claim 1, wherein the one or more metrics include an area under curve metric; and
wherein a first result for a first sample from the one or more samples is determined based on an area under a portion of a curve for the first sample that plots measurements for the first sample over time within the analysis window.

3. The computer-implemented method of claim 1, wherein the one or more metrics include a slope metric; and
wherein a first result for a first sample from the one or more samples is determined based on a slope for a portion of a curve for the first sample that plots measurements for the first sample over time within the analysis window.

4. The computer-implemented method of claim 1, wherein the one or more metrics include an area under curve per unit of time metric; and
wherein a first result for a first sample from the one or more samples is determined based on i) an area under a portion of a curve for the first sample that plots measurements for the first sample over time within the analysis window, and ii) divided the unit of time.

5. The computer-implemented method of claim 4, wherein the unit of time comprises an hour.

6. The computer-implemented method of claim 1, wherein a first waypoint from the waypoints is defined based on a time at which a treatment was applied to the one or more samples.

7. The computer-implemented method of claim 6, wherein a second waypoint from the waypoints is defined based on a period of time after the treatment was applied to the one or more samples.

8. The computer-implemented method of claim 1, wherein a first waypoint from the waypoints is defined based on a time interval after treatment was applied to the one or more samples.

9. The computer-implemented method of claim 8, wherein a second waypoint from the waypoints is defined based on a time at which the one or more samples reached a maximum cell index value.

10. The computer-implemented method of claim 8, wherein a second waypoint from the waypoints is defined based on a time at which one or more curves that are generated from and that correspond to the one or more samples had a maximum slope.

11. The computer-implemented method of claim 8, wherein a second waypoint from the waypoints is defined based on a time at which one or more curves that are generated from and that correspond to the one or more samples had a minimum slope.

12. The computer-implemented method of claim 1, further comprising smoothing the cellular data from the real time cell analyzer.

13. A computer system comprising:
one or more computing devices;
an interface of the one or more computing devices that is programmed to obtain cellular data from a real time cell analyzer;
a waypoint unit that is programmed i) to select one or more samples from the cellular data and ii) to identify, for each of the one or more samples or for the cellular data as a whole, waypoints that define an analysis window;
a quantitative analysis unit that is programmed to determine, using cellular data from the analysis window, results for each of the one or more samples based on one or more metrics; and
a data presentation unit that is programmed to provide information that identifies at least a portion of the results for the one or more samples.

14. The computer system of claim 13, wherein the one or more metrics include an area under curve metric; and
wherein a first result for a first sample from the one or more samples is determined by the quantitative analysis unit based on an area under a portion of a curve for the first sample that plots measurements for the first sample over time within the analysis window.

15. The computer system of claim 13, wherein the one or more metrics include a slope metric; and
wherein a first result for a first sample from the one or more samples is determined by the quantitative analysis unit based on a slope for a portion of a curve for the first sample that plots measurements for the first sample over time within the analysis window.

16. The computer system of claim 13, wherein the one or more metrics include an area under curve per unit of time metric; and
wherein a first result for a first sample from the one or more samples is determined by the quantitative analysis unit based on i) an area under a portion of a curve for the first sample that plots measurements for the first sample over time within the analysis window, and ii) divided the unit of time.

17. The computer system of claim 13, wherein a first waypoint from the waypoints is defined by the waypoint unit based on a time at which a treatment was applied to the one or more samples; and
wherein a second waypoint from the waypoints is defined by the waypoint unit based on a period of time after the treatment was applied to the one or more samples.

18. The computer system of claim 13, wherein a first waypoint from the waypoints is defined by the waypoint unit based on a time interval after treatment was applied to the one or more samples; and
wherein a second waypoint from the waypoints is defined by the waypoint unit based on one or more of:
a time at which the one or more samples reached a maximum cell index value,
a time at which one or more curves that are generated from and that correspond to the one or more samples had a maximum slope, and
a time at which one or more curves that are generated from and that correspond to the one or more samples had a minimum slope.

19. The computer system of claim 13, further comprising:
a data smoothing unit that is programmed to smooth the cellular data from the real time cell analyzer.

20. A computer program product embodied in a computer readable storage device storing instructions that, when executed, cause one or more computing devices to perform operations comprising:
obtaining cellular data from a real time cell analyzer;
selecting one or more samples from the cellular data;
identifying, for each of the one or more samples or for the cellular data as a whole, waypoints that define an analysis window;
determining, using cellular data from the analysis window, results for each of the one or more samples based on one or more metrics, the results indicating at least one characteristic of the cellular data within the analysis window; and
providing information that identifies at least a portion of the results for the one or more samples.
